(19) United States
(12) Patent Application Publication
(10) Pub. No.: US 2024/0085383 A1
(43) Pub. Date: Mar. 14, 2024

(54) METHOD FOR DETECTING CONTENT OF GLYCOSAMINOGLYCAN CARBOXYLATED DERIVATIVE IN SAMPLE, AND APPLICATION THEREOF

(71) Applicant: SHENZHEN HEPALINK PHARMACEUTICAL GROUP CO., LTD., Shenzhen, Guangdong (CN)

(72) Inventors: Jingwen WANG, Shenzhen, Guangdong (CN); Lige REN, Shenzhen, Guangdong (CN); Senmao LIN, Shenzhen, Guangdong (CN); Li LI, Shenzhen, Guangdong (CN)

(21) Appl. No.: 18/259,935

(22) PCT Filed: Dec. 30, 2021
(86) PCT No.: PCT/CN2021/142793

§ 371 Date: Jun. 29, 2023

(30) Foreign Application Priority Data
Dec. 31, 2020 (CN) 202011624408.8

(51) Int. Cl. G01N 30/72; G01N 1/44; G01N 30/12; G01N 30/88

(52) U.S. Cl. CPC G01N 30/7233; G01N 1/44; G01N 30/12; G01N 30/88; G01N 2030/067; G01N 2030/126; G01N 2030/884; G01N 2400/40

(57) ABSTRACT

The present application relates to a method for detecting the content of a glycosaminoglycan carboxylated derivative in a sample, and an application thereof. The method comprises: (1) hydrolyzing a sample to obtain a hydrolysate containing a compound as represented by formula (I); (2) testing the hydrolysate by means of liquid chromatography-tandem mass spectrometry; and (3) by using a glycosaminoglycan carboxylated derivative as a standard substance, hydrolyzing solutions thereof having different gradient concentrations according to the method in step (1), detecting, according to the method in step (2), mass spectrum signal peak areas of the compound as represented by formula (I) in the hydrolysates of the standard substance solutions having different concentrations, forming a standard curve on the basis of the mass spectrum signal peak areas against the amounts of the glycosaminoglycan carboxylated derivative standard substance, and according to the standard curve, calculating the content of the glycosaminoglycan carboxylated derivative in the sample according to the mass spectrum peak areas of the compound as represented by formula (I) determined in step (2). According to the method in the present application, hydrolysis products of a specific structure can be stably obtained by hydrolysis of the glycosaminoglycan carboxylated derivative, the structure can be detected by means of MS, and a hydrolysis product having higher mass spectrum abundance is selected, so that the amount of the glycosaminoglycan carboxylated derivative can be indirectly calculated. Moreover, the detection method has strong specificity, high accuracy, good precision, low limit of quantitation, and low limit of detection.

### FIELD OF THE INVENTION

The present application belongs to the technical field of analytical chemistry, and specifically relates to a method for detecting the content of a carboxylated glycosaminoglycan derivative in a sample and use thereof, and especially to a method for detecting the content of a carboxylated glycosaminoglycan derivative in a sample and use thereof with high specificity, high accuracy, good precision, low limit of quantitation and low limit of detection.

### BACKGROUND OF THE INVENTION

The prior art, such as patents CN105744940A and CN111670038A, reported that carboxylated glycosaminoglycan derivatives have anti-tumor and anti-metastasis activity as drugs, which have wide application prospects. The carboxylated glycosaminoglycan derivative is a bicarboxylic acid derivative obtained from unfractionated heparin (UFH) by a two-step oxidation reaction, where the uronic acid vicinal diol structure is oxidized and ring-opened, and the two-step oxidation reaction includes (1) two adjacent alcohol groups on the uronic acid of the glycosaminoglycan are oxidized and the ring is opened to form a dialdehyde structure, and (2) the dialdehyde structure is further oxidized to obtain the dicarboxylic acid structure; the carboxylated glycosaminoglycan derivative belongs to heparin derivatives, which is a linear and structurally inhomogeneous mucopolysaccharide substance. In drug metabolism studies, the drug content of biological samples is required to evaluate the pharmacokinetic properties of the drug. It is difficult to directly detect the intact structure in biological samples; additionally, due to the presence of endogenous substances in biological samples, such as proteins and phospholipids, conventional polysaccharide detection methods are susceptible to the interference of endogenous substances, and thus cannot quantitatively determine the carboxylated glycosaminoglycan derivative. Therefore, it has been an urgent problem to be solved about how to provide an accurate method for detecting a carboxylated glycosaminoglycan derivative.

### SUMMARY OF THE INVENTION

The following is a summary of the subject matter described in detail herein. The summary is not intended to limit the protection scope of the claims.

In view of the deficiencies of the prior art, an object of the present application is to provide a method for detecting the content of a carboxylated glycosaminoglycan derivative in a sample and use thereof, in particular to a method for detecting the content of a carboxylated glycosaminoglycan derivative in a sample and use thereof with high specificity, high accuracy, good precision, low limit of quantitation and low limit of detection.

To achieve the object, the present application adopts the technical solutions described below.

In a first aspect, the present application provides a method for detecting the content of a carboxylated glycosaminoglycan derivative in a sample. The method includes the following steps:
(1) hydrolyzing a sample containing a carboxylated glycosaminoglycan derivative to obtain a hydrolysate containing a compound of formula (I):

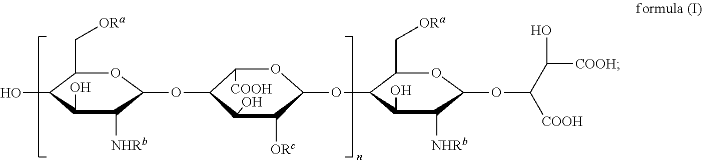

wherein, each R^{a} is independently —SO₃H or —H, each R^{b} is independently H, —SO₃H or —C(O)CH₃, each R^{c} is independently —SO₃H or —H, and n is 0, 1, 2, 3, 4 or 5;
(2) detecting the hydrolysate obtained in step (1) by liquid chromatography tandem mass spectrometry; and
(3) hydrolyzing solutions containing different gradient concentrations of the carboxylated glycosaminoglycan derivative as a standard according to the method of step (1); detecting mass spectral signal peak areas of the compound of formula (I) in the hydrolysates of the solutions containing different concentrations of the standard according to the method of step (2); establishing a standard curve between the mass spectral signal peak areas and the contents of the carboxylated glycosaminoglycan derivative standard, and calculating the content of the carboxylated glycosaminoglycan derivative in the sample based on the standard curve and the mass spectral signal peak area of the compound of formula (I) determined according to the method of step (2);
the carboxylated glycosaminoglycan derivative is a glycosaminoglycan compound including a structural unit of formula (II) and optionally a structural unit of formula (III):

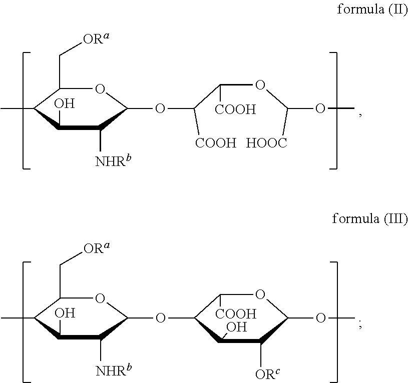

wherein, each R^{a} is independently —SO₃H or —H, R^{b} is independently H, —SO₃H or —C(O)CH₃, and R^{c} is independently —SO₃H or —H.

Preferably, the glycosaminoglycan is heparin or heparan sulfate; the carboxylated glycosaminoglycan derivative is obtained by a two-step oxidation reaction, including: (1) oxidizing two adjacent alcohol groups on the uronic acid of the glycosaminoglycan and ring-opening to form a dialdehyde structure, and (2) further oxidizing the dialdehyde structure to obtain a dicarboxylic acid structure.

Preferably, the compound of formula (I) has a structure of at least one of the following structural formulas:

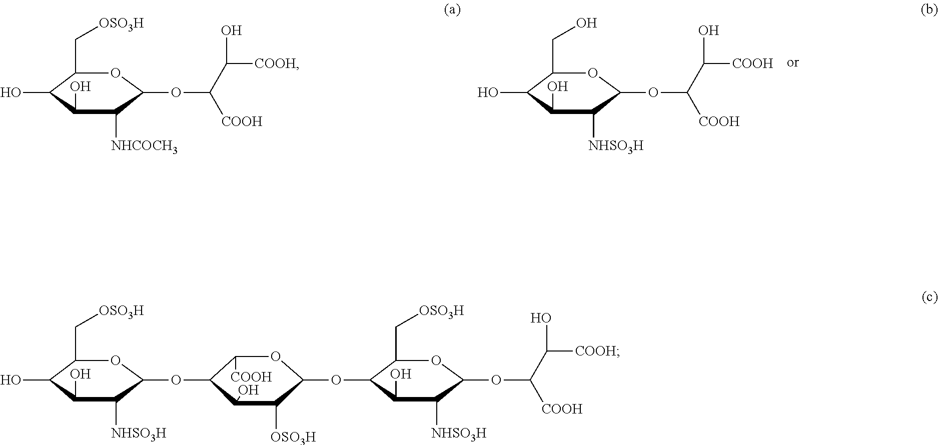

wherein, a mass spectral signal of compound (a) is MS (ESI, neg. ion) m/z: 432.0 [M−H]⁻;
a mass spectral signal of compound (b) is MS (ESI, neg. ion) m/z: 390.0 [M−H]⁻;
a mass spectral signal of compound (c) is MS (ESI, neg. ion) m/z: 522.98 [M−2H]²⁻.

The carboxylated glycosaminoglycan derivative involved in the present application includes the structural unit of formula (II) and optionally the structural unit of formula (III), that is, the hexuronic acid structure in the glycosaminoglycan compound is partially or completely ring-opened.

The carboxylated glycosaminoglycan derivative involved in the present application can be hydrolyzed to obtain the compound of formula (I), a reaction mechanism of which is shown in Schemes 1 and 2, wherein, each R^{a} is independently —SO₃H or —H, each R^{b} is independently H, —SO₃H or —C(O)CH₃, each R^{c} is independently —SO₃H or —H, and n is 0, 1, 2, 3, 4 or 5.

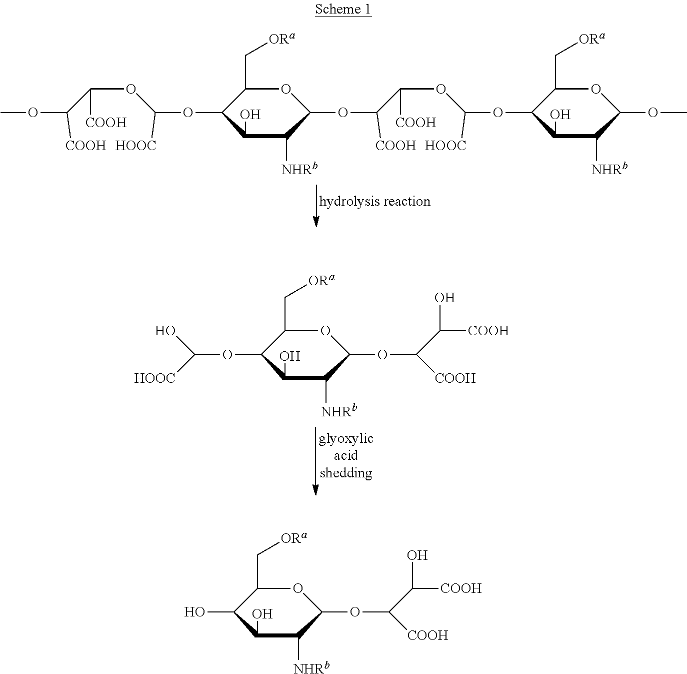

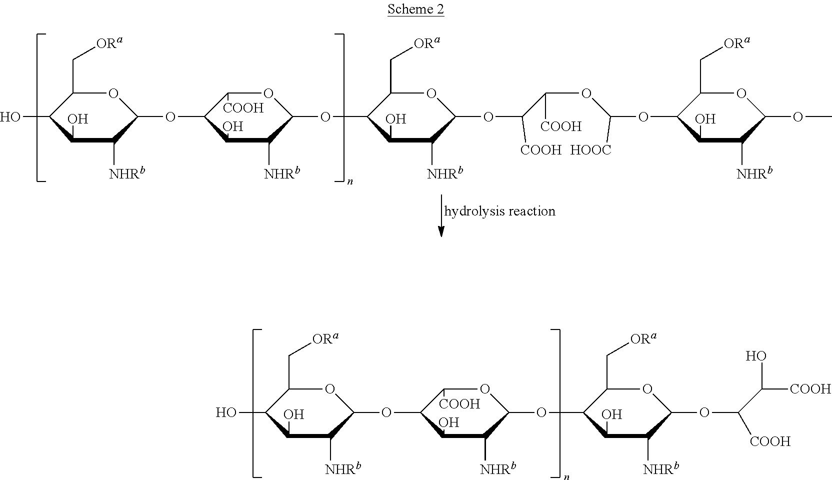

For each polysaccharide chain of the carboxylated glycosaminoglycan derivative involved in the present application, each disaccharide structural unit is arranged in any order.

Preferably, the carboxylated glycosaminoglycan derivative has a weight average molecular weight of 3000-20000 Da, such as 3000 Da, 5000 Da, 7000 Da, 8000 Da, 9000 Da, 10000 Da, 11000 Da, 12000 Da, 13000 Da, 13500 Da, 14000 Da, 16000 Da, 18000 Da or 20000 Da, etc. Other specific point values within this numerical range all can be selected and will not be listed here. The weight average molecular weight is preferably 7000-14000 Da, further preferably 8000-13500 Da.

The carboxylated glycosaminoglycan derivative has a ring-opening degree of 10-100%, such as 10%, 20%, 25%, 30%, 40%, 50%, 60%, 70%, 80%, 90% or 100%, etc. Other specific point values within this numerical range all can be selected and will not be listed here. The ring-opening degree is preferably 25-80%, further preferably 25-60%.

The term “ring-opening degree” in the present application refers to a ratio of the number of ring-opened uronic acid residues to the total number of uronic acid residues, which is detected and calculated with reference to the nuclear magnetic resonance method in the document: Guerrini, M., Guglieri, S, Naggi, A, Sasisekharan, R, (2007). Low molecular weight heparins: Structural differentiation by bidimensional nuclear magnetic resonance spectroscopy. Seminars in Thrombosis and Hemostasis, 33, 478-487.

Preferably, in step (1), a method of hydrolyzing the sample containing a carboxylated glycosaminoglycan derivative is heating.

Preferably, the heating is performed at 70-100° C., such as 70° C., 75° C., 80° C., 82° C., 85° C., 87° C., 95° C., 100° C., etc. Other specific point values within this numerical range all can be selected and will not be listed here. Preferably, the heating is performed at 85-95° C.

Preferably, the heating is performed for 12-168 h, such as 12 h, 16 h, 24 h, 36 h, 48 h, 55 h, 60 h, 70 h, 72 h, 75 h, 78 h, 80 h, 90 h, 96 h, 120 h, 144 h, or 168 h, etc. Other specific point values within this numerical range all can be selected and will not be listed here. Preferably, the heating is performed for 12-120 h.

Under the temperature and time conditions of heating, the carboxylated glycosaminoglycan derivative involved in the present application can produce compounds (a), compounds (b) or compounds (c) with high mass spectral abundance by hydrolysis, and the temperature and time of the reaction are selected with comprehensive consideration of detection efficiency, detection accuracy and precision.

When the sample containing a carboxylated glycosaminoglycan derivative is a biological sample (such as blood or urine), the hydrolysate obtained in step (1) requires detection pretreatment; the pretreatment includes: mixing the hydrolysate with a trifluoroacetic acid solution and an acetonitrile-methanol solution, then performing standing and centrifugation, collecting a supernatant and drying, and then re-dissolving with water.

Preferably, the trifluoroacetic acid solution is added, by volume, in an amount of 0.5-1.5% of the hydrolysate, such as 0.5%, 0.8%, 1.0%, 1.2% or 1.5%, etc. Other specific point values within this numerical range all can be selected and will not be listed here.

Preferably, the trifluoroacetic acid solution has a concentration of 4-6%, such as 4%, 5% or 6%, etc. Other specific point values within this numerical range all can be selected and will not be listed here.

Preferably, the acetonitrile-methanol solution is added, by volume, in an amount of 1-5 times of a volume of the hydrolysate, such as 1 time, 1.5 times, 1.8 times, 2.0 times, 2.2 times, 2.5 times, 3 times, 4 times, 5 times, etc. Other specific point values within this numerical range all can be selected and will not be listed here. The amount is preferably 1-3 times, more preferably 1.5-2.5 times.

Preferably, a volume ratio of acetonitrile to methanol in the acetonitrile-methanol solution is 1:0.5-1:1.5, such as 1:0.5, 1:0.8, 1:1, 1:1.2 or 1:1.5, etc. Other specific point values within this numerical range all can be selected and will not be listed here.

Preferably, the standing is performed at −25 to −15° C., such as −25° C., −20° C., −15° C., etc.; the standing is performed for 15-25 min, such as 15 min, 18 min, 20 min, 22 min, 25 min, etc. Other specific point values within the above numerical ranges all can be selected and will not be listed here.

Preferably, the liquid chromatography is reversed-phase chromatography, size-exclusion chromatography or hydrophilic chromatography.

Preferably, mobile phases of the liquid chromatography are mobile phase A and mobile phase B; the mobile phase A is an aqueous solution of hexafluoroisopropanol and pentylamine; the mobile phase B is an acetonitrile-water solution of hexafluoroisopropanol and pentylamine;
the mobile phase A is an aqueous solution containing 45-55 mM (such as 45 mM, 48 mM, 50 mM, 52 mM, 55 mM, etc.) hexafluoroisopropanol and 13-17 mM (such as 13 mM, 15 mM, 17 mM, etc.) pentylamine; the mobile phase B is an acetonitrile-water solution containing 45-55 mM (such as 45 mM, 48 mM, 50 mM, 52 mM, 55 mM, etc.) hexafluoroisopropanol and 13-17 mM (such as 13 mM, 15 mM, 17 mM, etc.) pentylamine; the mobile phase B has a volume ratio of acetonitrile to water of 70:30-80:20 (such as 70:30, 75:25, 80:20, etc.). Other specific point values within the above numerical ranges all can be selected and will not be listed here.

Further preferably, the mobile phases of the liquid chromatography are mobile phase A and mobile phase B, which is as below in the table.

| | |
|---|---|
| Mobile | 50 mM hexafluoroisopropanol, 15 mM pentylamine, H₂O |
| phase A | |
| Mobile | 50 mM hexafluoroisopropanol, 15 mM pentylamine, acetonitrile/ |
| phase B | H₂O (75/25, v/v) |

Preferably, the elution process of the liquid chromatography is as below in the table:

| Time (min) | Flow rate (mL/min) | % A | % B | Curve |
|---|---|---|---|---|
| Initial | 0.36 | 98.0 | 2.0 | Initial |
| 3.00 | 0.36 | 98.0 | 2.0 | 6 |
| 10.00 | 0.36 | 80.0 | 20.0 | 6 |
| 15.00 | 0.36 | 60.0 | 40.0 | 6 |
| 15.10 | 0.36 | 10.0 | 90.0 | 6 |
| 18.00 | 0.36 | 10.0 | 90.0 | 6 |
| 18.10 | 0.36 | 98.0 | 2.0 | 6 |
| 22.00 | 0.36 | 98.0 | 2.0 | 6 |

In the present application, the mass spectrometry conditions may exemplarily be the following conditions:

| Conditions | Name/Indicator | |
|---|---|---|
| Mass Spectrometer | Waters Xevo G2-S QTOF | |
| Mode | Negative Resolution Mode | |
| Set molecular weight | 432.0 | Da |
| Capillary voltage | 1.5 | kV |
| Sampling cone | 25 | V |
| Source compensation voltage | 80 | V |
| Source temperature | 120° | C. |
| Desolvation temperature | 500° | C. |
| Cone hole gas flow | 50 | L/Hr |
| Desolvation gas flow | 800 | L/Hr |
| Acquisition start molecular weight | 200 | |
| Acquisition termination molecular weight | 2000 | |
| Acquisition start time | 1.20 | min |
| Acquisition termination time | 6.0 | min |

Based on the first aspect, the present application also provides a new compound, and the specific content is as below.

In a second aspect, the present application provides a compound, and the compound has a structure of formula (I):

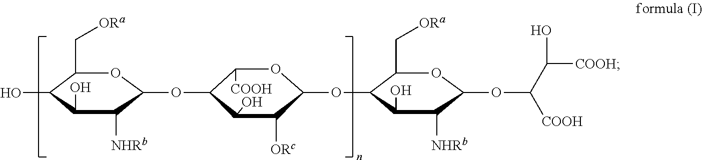

wherein, each R^{a} is independently —SO₃H or —H, each R^{b} is independently H, —SO₃H or —C(O)CH₃, each R^{c} is independently —SO₃H or —H, and n is 0, 1, 2, 3, 4 or 5.

Preferably, the compound has one of the following structures:

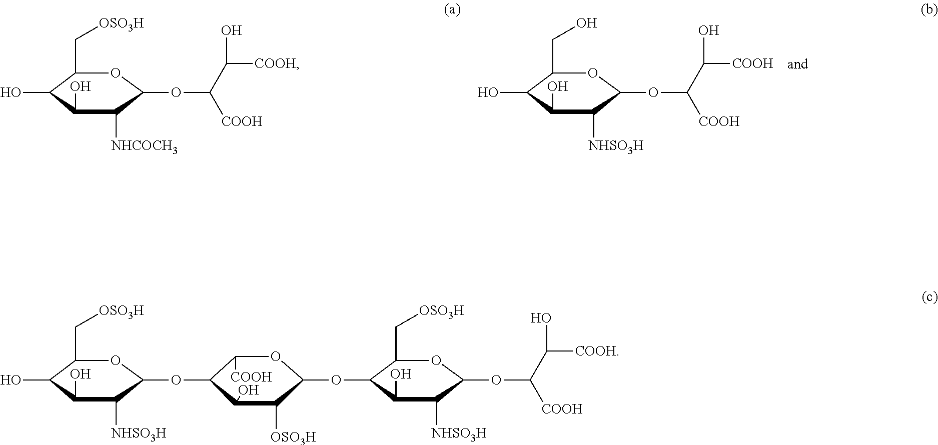

In a third aspect, the present application provides use of the method for detecting a carboxylated glycosaminoglycan derivative according to the first aspect in a pharmacokinetic study of a carboxylated glycosaminoglycan derivative.

In a fourth aspect, the present application provides use of the method for detecting a carboxylated glycosaminoglycan derivative according to the first aspect in a quality test of a carboxylated glycosaminoglycan derivative pharmaceutical preparation.

Compared with the prior art, the present application has the following beneficial effects.

Because the carboxylated glycosaminoglycan derivative is an inhomogeneous substance, its intact structure is difficult to be detected directly in biological samples. It is found by the inventors of the present application that the carboxylated glycosaminoglycan derivative can be hydrolyzed to reliably produce hydrolysis products compound (a), compound (b) or compound (c) with specific structures as described above, and such compounds can be detected by mass spectrometry. By detecting compounds (a), compounds (b) or compounds (c), a standard curve is established with different concentrations of the carboxylated glycosaminoglycan derivative standard and the corresponding mass spectral peak areas of compounds (a), compounds (b) or compounds (c); then the carboxylated glycosaminoglycan derivative in a sample is hydrolyzed and mass spectral peak areas of compounds (a), compounds (b) or compounds (c) are detected; the sample containing the carboxylated glycosaminoglycan derivative is hydrolyzed and then detecting the mass spectral peak areas of compounds (a), compounds (b) or compounds (c) by liquid chromatography tandem mass spectrometry, and the content of the carboxylated glycosaminoglycan derivative in the sample can be indirectly calculated out based on the standard curve. The detection method has high specificity, high accuracy, good precision, low limit of quantitation and low limit of detection.

After reading and understanding the detailed description, other aspects can be understood.

### BRIEF DESCRIPTION OF THE DRAWING

FIG. 1 is a mass spectrum of compound (a);

FIG. 2 is a secondary mass spectrum of compound (a);

FIG. 3 is a ¹H-NMR spectrum of compound (a);

FIG. 4 is a ¹³C-NMR spectrum of compound (a);

FIG. 5 is a ¹³C DEPT 135° NMR spectrum of compound (a);

FIG. 6 is a ¹H-¹H COSY spectrum of compound (a);

FIG. 7 is a TOCSY spectrum of compound (a);

FIG. 8 is an HSQC spectrum of compound (a);

FIG. 9 is an HMBC spectrum of compound (a);

FIG. 10 is a mass spectrum of compound (c);

FIG. 11 is a secondary mass spectrum of compound (c);

FIG. 12 is a ¹H-NMR spectrum of compound (c);

FIG. 13 is a ¹³C-NMR spectrum of compound (c);

FIG. 14 is a ¹³C DEPT 135° NMR spectrum of compound (c);

FIG. 15 is a ¹H-¹H COSY spectrum of compound (c);

FIG. 16 is a TOCSY spectrum of compound (c);

FIG. 17 is a ROESY spectrum of compound (c);

FIG. 18 is an HSQC spectrum of compound (c); and

FIG. 19 is an HMBC spectrum of compound (c).

### DETAILED DESCRIPTION OF THE INVENTION

The technical solutions of the present application are further described below through examples. It should be apparent to those skilled in the art that the examples are only used for a better understanding of the present application and should not be construed as a specific limitation of the present application.

The SD rats involved in the following examples were purchased from Beijing Vital River Laboratory Animal Technology Co., Ltd.

The carboxylated glycosaminoglycan derivative H1011 involved in the following examples was prepared by the preparation method disclosed in Example 3 of patent CN111670038A, which has a weight average molecular weight of 9161 Da and a ring-opening degree of 43.1%.

### EXAMPLE 1

### Preparation of Compound (a), Compound (b) and Compound (c)

The H1011 (400 mg) was weighed out and dissolved in water (4.0 mL), and the aqueous solution of H1011 was heated to 85° C. and reacted for 72 h and then cooled to 25° C.; the reaction products were purified and separated by chromatographic technique (chromatographic column: Dionex IonPac AS11-HC, eluent: M and N (see Table 1 for specific components)), and then subjected to desalination and lyophilization to obtain compound (a), compound (b) and compound (c).

TABLE 1

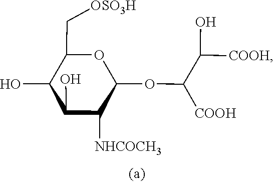

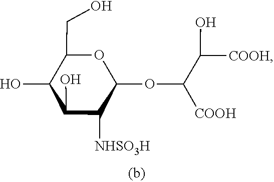

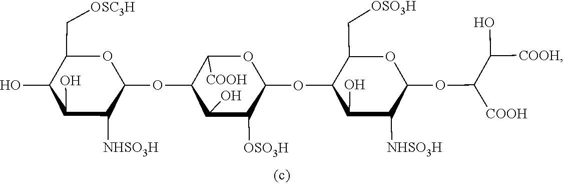

Table 1

| | Eluent M (%) | Eluate N (%) |
|---|---|---|
| Time | (2.33 mM sodium dihydrogen | (2.33 mM sodium dihydrogen phosphate, |
| (min) | phosphate) | 1.14 M sodium perchlorate) |
| 0 | 97 | 3 |
| 8 | 91 | 9 |
| 20 | 72 | 28 |
| 25 | 0 | 100 |
| 25.1 | 97 | 3 |
| 30 | 97 | 3 |

The compound (a), compound (b) and compound (c) obtained by enrichment were structurally identified by mass spectrometry and nuclear magnetic resonance spectroscopy (1D ¹H-NMR 1D ¹³C-NMR, ¹³C DEPT 135°, ¹H-¹H COSY, 2D TOCSY, HSQC, HMBC, 2D DOSY).

The characterization result for compound (a) is as follows.

MS (ESI, neg. ion) m/z: 432.0 [M−H]⁻.

¹H-NMR (600 MHz, D₂O): δ 4.87 (d, J=3.5 Hz, 1H), 4.34 (dd, J=11.2, 3.7 Hz, 1H), 4.29 (d, J=4.6 Hz, 1H), 4.27 (dd, J=11.0, 2.1 Hz, 1H), 4.20 (d, J=4.6 Hz, 1H), 4.05 (ddd, J=10.1, 3.7, 2.2 Hz, 1H), 3.96 (dd, J=10.6, 3.5 Hz, 1H), 3.81 (dd, J=10.5, 9.2 Hz, 1H), 3.59 (dd, J=10.1, 9.2 Hz, 1H), 2.07 (s, 3H).

¹³C-NMR (151 MHz, D₂O): δ 24.79, 56.23, 69.39, 71.95, 73.04, 74.14, 76.51, 83.25, 99.56, 177.34, 178.79, 179.99.

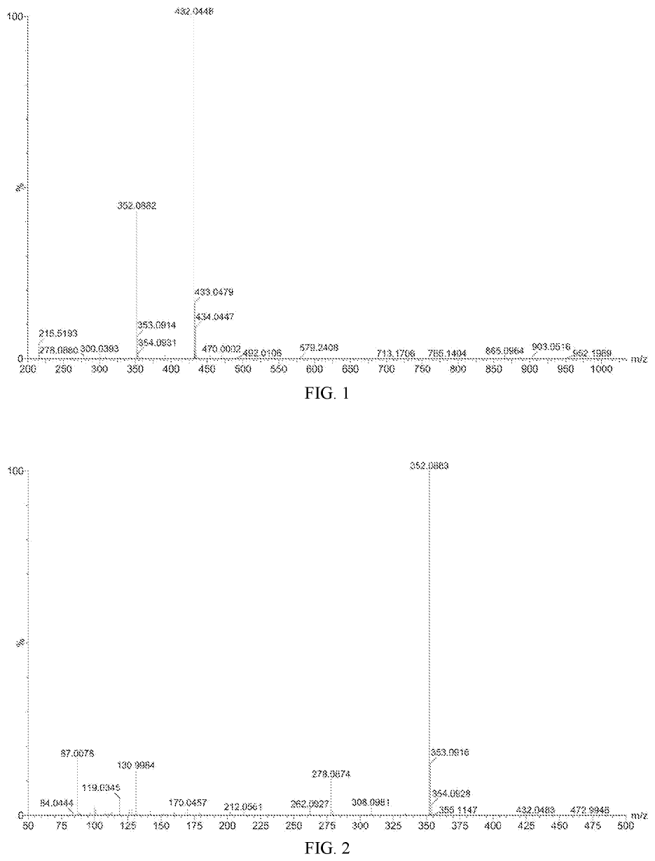

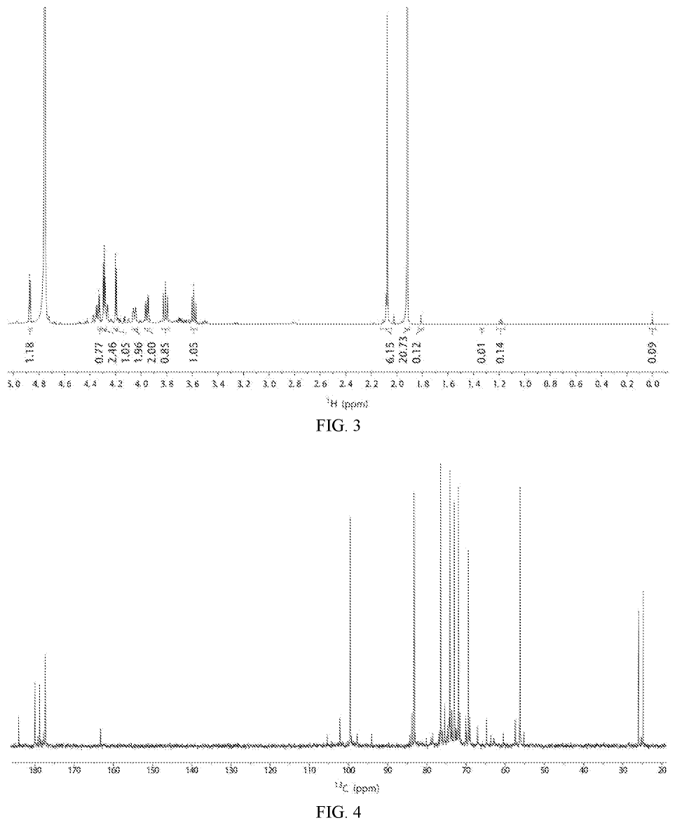

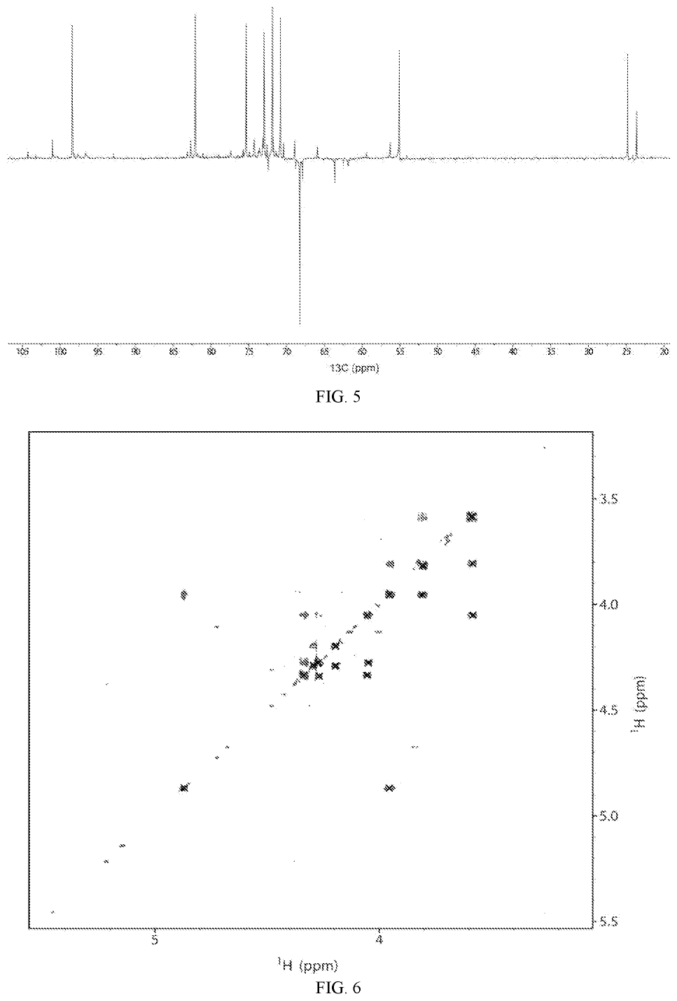

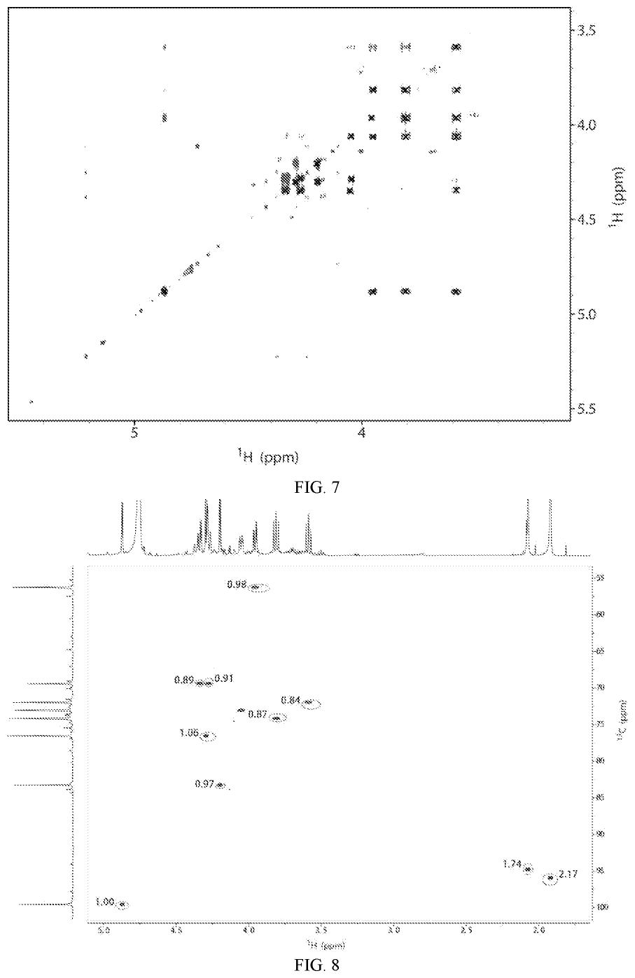

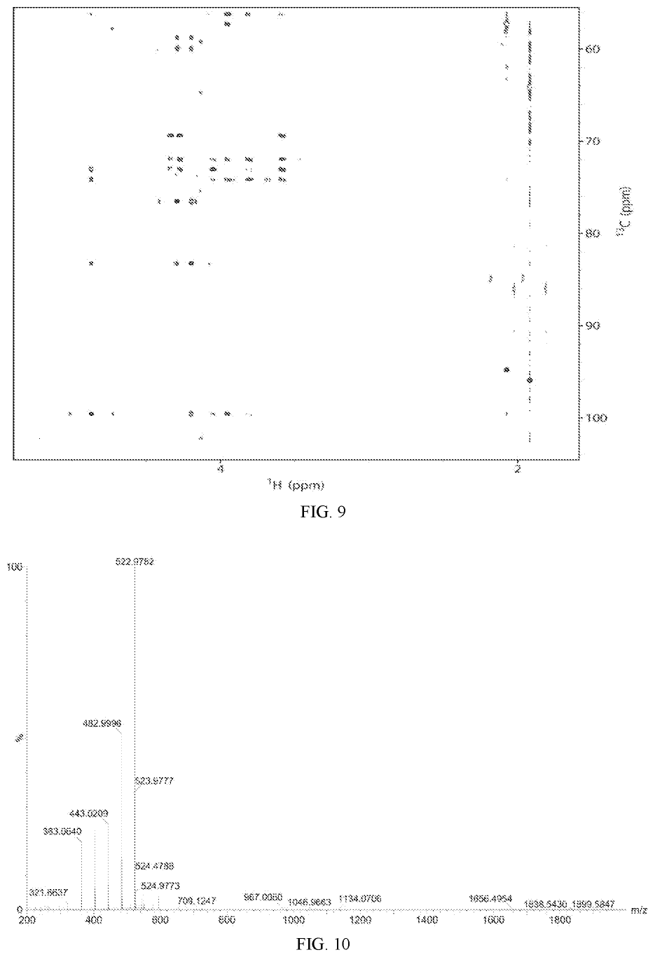

The characterization spectra of compound (a) are shown in FIGS. 1-9.

The characterization result for compound (b) is as follows.

MS (ESI, neg. ion) m/z: 390.0 [M−H]⁻.

The characterization result for compound (c) is as follows.

MS (ESI, neg. ion) m/z: 522.98 [M−2H]²⁻.

¹H-NMR (600 MHz, D₂O): δ 5.37 (d, J=3.5 Hz, 1H), 5.25-5.23 (m, 1H), 5.16 (d, J=3.6 Hz, 1H), 5.15-5.12 (m, 1H), 4.38-4.28 (m, 5H), 4.23 (dd, J=11.4, 2.1 Hz, 1H), 4.19 (dd, J=11.1, 2.1, 1H), 4.13 (dd, J=2.9 Hz, 1H), 3.87 (dt, J=9.7, 2.7 Hz, 1H), 3.84 (dt, J=9.9, 2.6 Hz, 1H), 3.78-3.70 (m, 2H), 3.64 (q, J=7.1 Hz, 1H), 3.60 (dd, J=9.9 Hz, 1H), 3.56 (dd, J=9.5 Hz, 1H), 3.25 (dd, J=10.1, 3.5 Hz, 3H).

¹³C-NMR (151 MHz, D₂O): δ 60.45, 60.63, 68.92, 69.10, 70.44, 70.60, 71.82, 72.44, 72.80, 73.05, 73.50, 74.54, 77.54, 78.74, 79.51, 81.24, 100.90, 101.38, 101.69, 175.69, 176.18, 177.23.

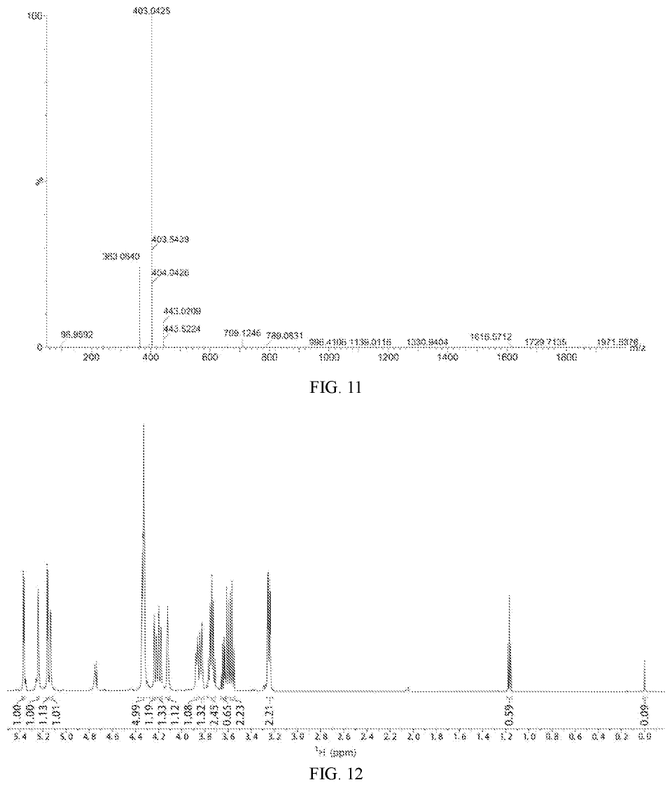

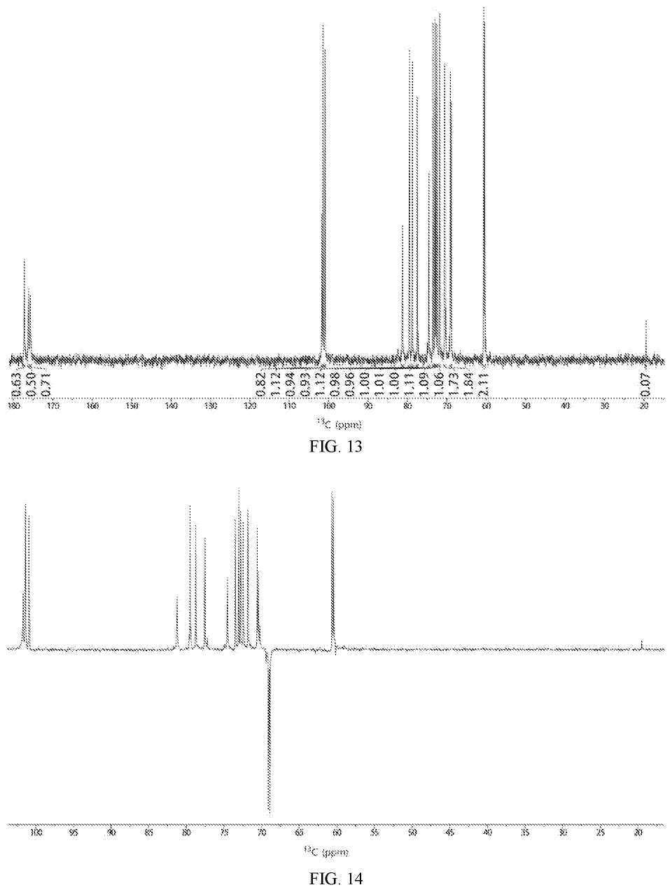

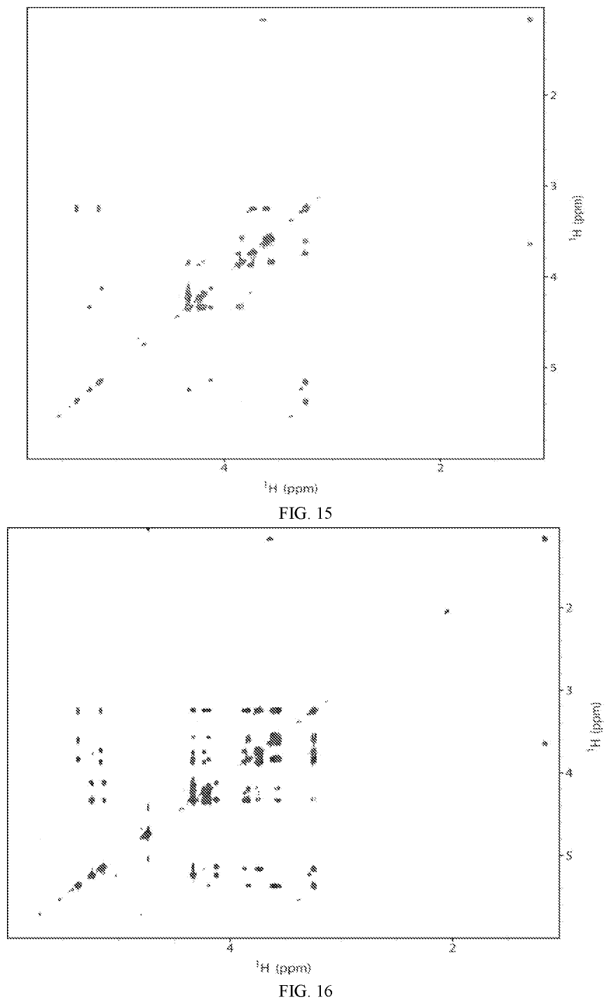

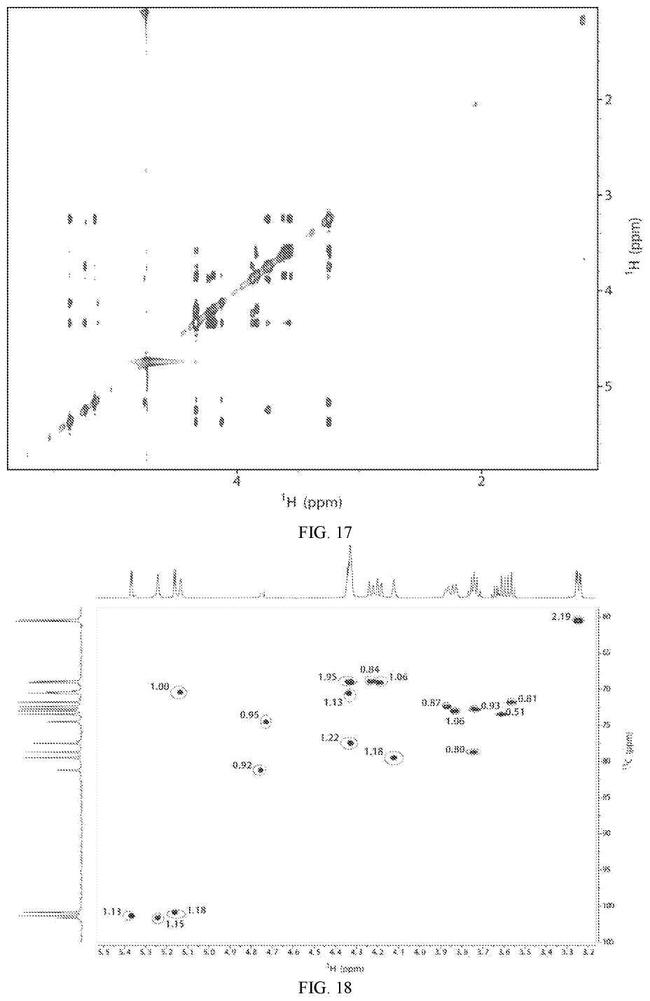

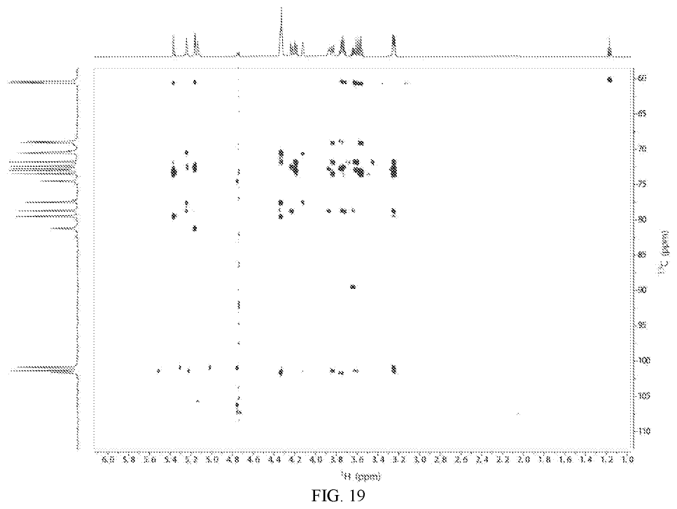

The characterization spectra of compound (c) are shown in FIGS. 10-19.

### EXAMPLE 2

This example applies the method for detecting a carboxylated glycosaminoglycan derivative involved in the present application to a pharmacokinetic study (with compound (a) as the detection object), and the specific contents are as follows.

### (1) Test Method

(1.1) Experimental animals: 6 healthy adult male SD rats, in which 3 rats were used to collect blank plasma to establish the standard curve and 3 rats were used to perform the plasma drug concentration detection after a single dose in rats.
(1.2) Drug preparation: the carboxylated glycosaminoglycan derivative H1011 was weighed out and prepared into a 60 mg/kg drug solution with water.
(1.3) Administration and sample collection: after administered by subcutaneous injection at a dose of 60 mg/kg, blood was collected at 0, 0.25, 0.5, 1, 2, 4, 6, 8 and 24 h points in time; whole blood was collected, than added in a K2EDTA anticoagulant tube, then centrifuged for 15 min, and separated to obtain a plasma sample.

### (1.4) Standard Curve Establishment:

(1.4.1) The carboxylated glycosaminoglycan derivative H1011 was weighed out and prepared into a 1 mg/mL aqueous solution, and the H1011 aqueous solution was diluted step by step with the blank plasma as a diluent to prepare 2 μg/mL, 4 μg/mL, 8 μg/mL, 16 μg/mL, 32 μg/mL, 64 μg/mL, 128 μg/mL, and 256 μg/mL standard solutions, and then hydrolyzed at 85° C. for 72 h.
(1.4.2) The hydrolyzed standard solution was pre-treated before the detection; the hydrolyzed standard solution was added with one percent volume of trifluoroacetic acid (5%, v/v) and two times volume of acetonitrile/methanol (v/v, 50/50), mixed uniformly, then allowed to stand at −20° C. for 20 min and centrifuged; a supernatant was collected, dried, and then re-dissolved with ultrapure water.
(1.4.3) The liquid chromatography tandem mass spectrometry detection was performed to obtain the spectrum; the chromatography conditions are shown in Table 2 and the mass spectrometry conditions are shown in Table 3.

TABLE 2

| Conditions | Name/Indicator | | | | |
|---|---|---|---|---|---|
| Ultra high | Waters ACQUITY UPLC | | | | |
| performance | | | | | |
| liquid | | | | | |
| chromatograph | | | | | |
| Detector/Detection | Waters TUV detector/232 nm | | | | |
| wavelength | | | | | |
| Chromatography | Waters Acquity UPLC BEH C18 1.7 um (2.1 mm × 150 mm) | | | | |
| column | | | | | |
| Mobile phase | Mobile phase A: 50 mM HFIP (hexafluoroisopropanol), 15 mM PTA | | | | |
| | (pentylamine), H₂O; | | | | |
| | Mobile phase B: 50 mM HFIP | | | | |
| | (hexafluoroisopropanol), 15 mM PTA (pentylamine), acetonitrile/H₂O (75/25, v/v); | | | | |
| Mobile phase | Time | Flow rate | | | |
| gradient | (min) | (mL/min) | % A | % B | Curve |
| | Initial | 0.36 | 98.0 | 2.0 | Initial |
| | 3.00 | 0.36 | 98.0 | 2.0 | 6 |
| | 10.00 | 0.36 | 80.0 | 20.0 | 6 |
| | 15.00 | 0.36 | 60.0 | 40.0 | 6 |
| | 15.10 | 0.36 | 10.0 | 90.0 | 6 |
| | 18.00 | 0.36 | 10.0 | 90.0 | 6 |
| | 18.10 | 0.36 | 98.0 | 2.0 | 6 |
| | 22.00 | 0.36 | 98.0 | 2.0 | 6 |

| | |
|---|---|
| Injection volume | 5 μL |
| Acquisition time | 22 min |
| Workstation | MassLynx v4.1 |

TABLE 3

| Conditions | Name/Indicator | |
|---|---|---|
| Mass Spectrometer | Waters Xevo G2-S QTOF | |
| Mode | Negative Resolution Mode | |
| Set molecular weight | 432.0 | Da |
| Capillary voltage | 1.5 | kV |
| Sampling cone | 25 | V |
| Source compensation voltage | 80 | V |
| Source temperature | 120° | C. |
| Desolvation temperature | 500° | C. |
| Cone hole gas flow | 50 | L/Hr |
| Desolvation gas flow | 800 | L/Hr |
| Acquisition start molecular weight | 200 | |
| Acquisition termination | 2000 | |
| molecular weight | | |
| Acquisition start time | 1.20 | mins |
| Acquisition termination time | 7.0 | mins |

(1.4.4) The standard curve was established according to the linear relationship between the H1011 standard solution and the mass spectral peak area of compound (a); the mass spectral signal of compound (a) is MS (ESI, neg. ion) m/z: 432.0 [M−H]⁻; the linear equation is: y=36.2154x−0.3216; the correlation coefficient is: R²=0.9987, wherein y is the mass spectral peak area and x is the concentration of the standard solution.

### (1.5) Detection of the Content of H1011 in the Plasma Sample

(1.5.1) The plasma sample obtained in step (1.3) was hydrolyzed at 85° C. for 72 h, then added with one percent volume of trifluoroacetic acid (5%, v/v) and two times volume of acetonitrile/methanol (v/v, 50/50), mixed uniformly, then allowed to stand at −20° C. for 20 min and centrifuged; a supernatant was collected, dried, and then re-dissolved with ultrapure water.
(1.5.2) The liquid chromatography tandem mass spectrometry detection was performed to obtain the mass spectral peak area of compound (a); the chromatography conditions are shown in Table 2 and the mass spectrometry conditions are shown in Table 3.
(1.5.3) Based on the mass spectral peak area of compound (a) and the standard curve established in step (1.4), the concentration of H1011 in the plasma sample at each time point was calculated, and pharmacokinetic parameters were calculated based on the drug concentration-time curve.
(2) The test result is shown in Table 4.

TABLE 4

| Administration | | AUC₀₋₂₄ | T_{1/2} | CL |
|---|---|---|---|---|
| route | Dosage | (h*μg/mL) | (h) | (mL/min/kg) |
| i.h. | 60 mg/kg | 537.47 | 3.75 | 0.110 |

### (3) Methodological Validation

### (3.1) Specificity

A ultrapure water control solution and a blank plasma control solution were prepared and processed by the same high-temperature hydrolysis and pretreatment procedure as described above, and then subjected to the liquid chromatography tandem mass spectrometry and detected under the same conditions as described above; no signal peak of 432.0 Da, i.e., the mass spectral signal peak of compound (a), was observed in the ultrapure water control solution and plasma control solution, indicating that ultrapure water and blank plasma have no interference to the detection, and the detection method has high specificity.

### (3.2) Limit of Quantitation and Limit of Detection

The limit of quantitation of the method was calculated to be 0.8 μg/mL and the limit of detection was 0.2 μg/mL.

### (3.3) Accuracy

The H1011 plasma samples with three concentrations designed as 1 μg/mL, 50 μg/mL and 120 μg/mL were detected; the recovery rate was calculated to be 82.6-110.8%, and the RSD values of 6 experimental results for each concentration were 4.2%, 2.2% and 1.3% in order.

### (3.4) Precision

The 50 μg/mL H1011 plasma solution was selected and detected by two different operators 6 times individually; the RSD of the 6 experimental results of the first operator was 2.0%, the RSD of the 6 experimental results of the second operator was 1.8%, and the RSD of the 12 experimental results of the two operators was 2.1%, all of which meet the acceptable criteria of less than or equal to 10.0%. The method has good precision.

### (3.5) Solution Stability

The 50 μg/mL H1011 plasma solution was selected, hydrolyzed and pretreated; the detection result of the sample solution at day 5 is 97.2% of the result at day 0, which meets the criteria.

### EXAMPLE 3

This example applies the method for detecting a carboxylated glycosaminoglycan derivative involved in the present application to a pharmacokinetic study (with compound (b) as the detection object), and the specific contents are as follows.

### (1) Test Method

(1.1) Experimental animals: 6 healthy adult male SD rats, in which 3 rats were used to collect blank plasma to establish the standard curve and 3 rats were used to perform the plasma drug concentration detection after a single dose in rats.
(1.2) Drug preparation: the carboxylated glycosaminoglycan derivative H1011 was weighed out and prepared into a 60 mg/kg drug solution with water.
(1.3) Administration and sample collection: after administered by subcutaneous injection at a dose of 60 mg/kg, blood was collected at 0, 0.25, 0.5, 1, 2, 4, 6, 8 and 24 h points in time; whole blood was collected, than added in a K2EDTA anticoagulant tube, then centrifuged for 15 min, and separated to obtain a plasma sample.

### (1.4) Standard Curve Establishment

(1.4.1) The carboxylated glycosaminoglycan derivative H1011 was weighed out and prepared into a 1 mg/mL aqueous solution, and the H1011 aqueous solution was diluted step by step with the blank plasma as a diluent to prepare 2 μg/mL, 4 μg/mL, 8 μg/mL, 16 μg/mL, 32 μg/mL, 64 μg/mL, 128 μg/mL and 256 μg/mL standard solutions, and then hydrolyzed at 90° C. for 48 h.
(1.4.2) The hydrolyzed standard solution was pre-treated before detection; the hydrolyzed standard solution was added with one percent volume of trifluoroacetic acid (5%, v/v) and two times volume of acetonitrile/methanol (v/v, 50/50), mixed uniformly, then allowed to stand at −20° C. for 20 min and centrifuged; a supernatant was collected, dried, and then re-dissolved with ultrapure water.
(1.4.3) The liquid chromatography tandem mass spectrometry detection was performed to obtain the spectrum; the chromatography conditions are shown in Table 2, the mass spectrometry conditions are shown in Table 3, and the molecular weight is set to be 390.0 Da.
(1.4.4) The standard curve was established according to the linear relationship between the H1011 standard solution and the mass spectral peak area of compound (b); the mass spectral signal of compound (b) is MS (ESI, neg. ion) m/z: 390.0 [M−H]⁻; the linear equation is: y=26.0235x; the correlation coefficient is: R²=0.9981, in which y is the mass spectral peak area and x is the concentration of the standard solution.

### (1.5) Detection of the Content of H1011 in the Plasma Sample

(1.5.1) The plasma sample obtained in step (1.3) was hydrolyzed at 90° C. for 48 h, then added with one percent volume of trifluoroacetic acid (5%, v/v) and two times volume of acetonitrile/methanol (v/v, 50/50), mixed uniformly, then allowed to stand at −20° C. for 20 min and centrifuged; a supernatant was collected, dried, and then re-dissolved with ultrapure water.
(1.5.2) The liquid chromatography tandem mass spectrometry detection was performed to obtain the mass spectral peak area of compound (b); the chromatography conditions are shown in Table 2, the mass spectrometry conditions are shown in Table 3, and the molecular weight is set to be 390.0 Da.
(1.5.3) Based on the mass spectral peak area of compound (b) and the standard curve established in step (1.4), the concentration of H1011 in the plasma sample at each time point was calculated, and pharmacokinetic parameters were calculated based on the drug concentration-time curve.
(2) The test result is shown in Table 5.

TABLE 5

| Administration | | AUC₀₋₂₄ | T_{1/2} | CL |
|---|---|---|---|---|
| route | Dosage | (h*μg/mL) | (h) | (mL/min/kg) |
| i.h. | 60 mg/kg | 483.87 | 3.04 | 0.089 |

### (3) Methodological Validation

### (3.1) Specificity

A ultrapure water control solution and a blank plasma control solution were prepared and processed by the same high-temperature hydrolysis and pretreatment procedure as described above, and then subjected to the liquid chromatography tandem mass spectrometry and detected under the same conditions as described above; no signal peak of 390.0 Da, i.e., the mass spectral signal peak of compound (b), was observed in the ultrapure water control solution and plasma control solution, indicating that ultrapure water and blank plasma have no interference to the detection, and the detection method has high specificity.

### (3.2) Limit of Quantitation and Limit of Detection

The limit of quantitation of the method was calculated to be 1.1 μg/mL and the limit of detection was 0.55 μg/mL.

### (3.3) Accuracy

The H1011 plasma samples with three concentrations designed as 1 μg/mL, 50 μg/mL and 120 μg/mL were detected; the recovery rate was calculated to be 81.2-115.8%, and the RSD values of 6 experimental results for each concentration were 5.4%, 1.8% and 2.0% in order.

### (3.4) Precision

The 50 μg/mL H1011 plasma solution was selected and detected by two different operators 6 times individually; the RSD of the 6 experimental results of the first operator was 1.6%, the RSD of the 6 experimental results of the second operator was 2.6%, and the RSD of the 12 experimental results of the two operators was 2.2%, all of which meet the acceptable criteria of less than or equal to 10.0%. The method has good precision.

### (3.5) Solution Stability

The 50 μg/mL H1011 plasma solution was selected, hydrolyzed and pretreated; the detection result of the sample solution at day 5 is 97.8% of the result at day 0, which meets the criteria.

### EXAMPLE 4

This example applies the method for detecting a carboxylated glycosaminoglycan derivative involved in the present application to a pharmacokinetic study (with compound (c) as the detection object), and the specific contents are as follows.

### (1) Test Method

(1.1) Experimental animals: 6 healthy adult male SD rats, in which 3 rats were used to collect blank plasma to establish the standard curve and 3 rats were used to perform the plasma drug concentration detection after a single dose in rats.
(1.2) Drug preparation: the carboxylated glycosaminoglycan derivative H1011 was weighed out and prepared into a 20 mg/kg drug solution with water.
(1.3) Administration and sample collection: after administered by subcutaneous injection at a dose of 60 mg/k, blood was collected at 0, 0.25, 0.5, 1, 2, 4, 6, 8 and 24 h points in time; whole blood was collected, than added in a K2EDTA anticoagulant tube, then centrifuged for 15 min, and separated to obtain a plasma sample.

### (1.4) Standard Curve Establishment

(1.4.1) The carboxylated glycosaminoglycan derivative H1011 was weighed out and prepared into a 1 mg/mL aqueous solution, and the H1011 aqueous solution was diluted step by step with the blank plasma as a diluent to prepare 2 μg/mL, 4 μg/mL, 8 μg/mL, 16 μg/mL, 32 μg/mL, 64 μg/mL, 128 μg/mL, and 256 μg/mL standard solutions, and then hydrolyzed at 90° C. for 36 h.
(1.4.2) The hydrolyzed standard solution was pre-treated before detection; the hydrolyzed standard solution was added with one percent volume of trifluoroacetic acid (5%, v/v) and two times volume of acetonitrile/methanol (v/v, 50/50), mixed uniformly, then allowed to stand at −20° C. for 20 min and centrifuged; a supernatant was collected, dried, and then re-dissolved with ultrapure water.
(1.4.3) The liquid chromatography tandem mass spectrometry detection was performed to obtain the spectrum; the chromatography conditions are shown in Table 2, the mass spectrometry conditions are shown in Table 3, and the molecular weight is set to be 522.98 Da.
(1.4.4) The standard curve was established according to the linear relationship between the H1011 standard solution and the mass spectral peak area of compound (c); the mass spectral signal of compound (c) is MS (ESI, neg. ion) m/z: 522.98 [M−2H]²⁻; the linear equation is: y=23.6225x; the correlation coefficient is: R²=0.9986, in which y is the mass spectral peak area and x is the concentration of the standard solution.

### (1.5) Detection of the Content of H1011 in the Plasma Sample

(1.5.1) The plasma sample obtained in step (1.3) was hydrolyzed at 90° C. for 36 h, then added with one percent volume of trifluoroacetic acid (5%, v/v) and two times volume of acetonitrile/methanol (v/v, 50/50), mixed uniformly, then allowed to stand at −20° C. for 20 min and centrifuged; a supernatant was collected, dried, and then re-dissolved with ultrapure water.
(1.5.2) The liquid chromatography tandem mass spectrometry detection was performed to obtain the mass spectral peak area of compound (c); the chromatography conditions are shown in Table 2 and the mass spectrometry conditions are shown in Table 3.
(1.5.3) Based on the mass spectral peak area of compound (c) and the standard curve established in step (1.4), the concentration of H1011 in the plasma sample at each time point was calculated, and pharmacokinetic parameters were calculated based on the drug concentration-time curve.
(2) The test result is shown in Table 6.

TABLE 6

| Administration | | AUC₀₋₂₄ | T_{1/2} | CL |
|---|---|---|---|---|
| route | Dosage | (h*μg/mL) | (h) | (mL/min/kg) |
| i.h. | 20 mg/kg | 237.44 | 4.04 | 0.073 |

### (3) Methodological Validation

### (3.1) Specificity

A ultrapure water control solution and a blank plasma control solution were prepared and processed by the same high-temperature hydrolysis and pretreatment procedure as described above, and then subjected to the liquid chromatography tandem mass spectrometry and detected under the same conditions as described above; no signal peak of 522.98 Da, i.e., the mass spectral signal peak of compound (c), was observed in the ultrapure water control solution and plasma control solution, indicating that ultrapure water and blank plasma have no interference to the detection, and the detection method has high specificity.

### (3.2) Limit of Quantitation and Limit of Detection

The limit of quantitation of the method was calculated to be 2.0 μg/mL and the limit of detection was 1.0 μg/mL.

### (3.3) Accuracy

The H1011 plasma samples with three concentrations designed as 2 μg/mL, 50 μg/mL and 120 μg/mL were detected; the recovery rate was calculated to be 80.3-108.8%, and the RSD values of 6 experimental results for each concentration were 8.9%, 6.5% and 5.4% in order.

### (3.4) Precision

The 50 μg/mL H1011 plasma solution was selected and detected by two different operators 6 times individually; the RSD of the 6 experimental results of the first operator was 6.5%, the RSD of the 6 experimental results of the second operator was 7.2%, and the RSD of the 12 experimental results of the two operators was 7.4%, all of which meet the acceptable criteria of less than or equal to 10.0%. The method has good precision.

### (3.5) Solution Stability

The 50 μg/mL H1011 plasma solution was selected, hydrolyzed and pretreated; the detection result of the sample solution at day 5 is 90.2% of the result at day 0, which meets the criteria.

1. A method for detecting the content of a carboxylated glycosaminoglycan derivative in a sample, comprising the following steps:
(1) hydrolyzing a sample containing a carboxylated glycosaminoglycan derivative to obtain a hydrolysate containing a compound of formula (I): 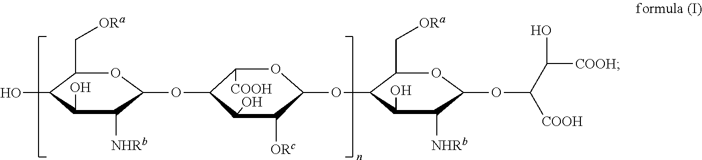
wherein, each R^{a} is independently —SO₃H or —H, each R^{b} is independently H, —SO₃H or —C(O)CH₃, each R^{c} is independently —SO₃H or —H, and n is 0, 1, 2, 3, 4 or 5;
(2) detecting the hydrolysate obtained in step (1) by liquid chromatography tandem mass spectrometry; and
(3) hydrolyzing solutions containing different gradient concentrations of the carboxylated glycosaminoglycan derivative as a standard according to the method of step (1); detecting mass spectral signal peak areas of the compound of formula (I) in the hydrolysates of the solutions containing different concentrations of the standard according to the method of step (2); establishing a standard curve between the contents of the carboxylated glycosaminoglycan derivative standard and the mass spectral signal peak areas; and calculating the content of the carboxylated glycosaminoglycan derivative in the sample based on the standard curve and the mass spectral signal peak area of the compound of formula (I) determined according to the method of step (2);
wherein, the carboxylated glycosaminoglycan derivative is a glycosaminoglycan compound comprising a structural unit of formula (II) and optionally a structural unit of formula (III): 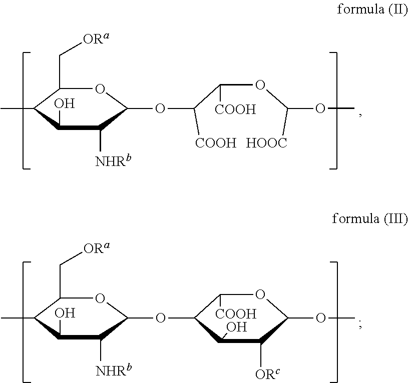
wherein, each R^{a} is independently —SO₃H or —H, R^{b} is independently H, —SO₃H or —C(O)CH₃, and R^{c} is independently —SO₃H or —H.

2. The method of claim 1, wherein
the carboxylated glycosaminoglycan derivative is obtained by a two-step oxidation reaction, comprising: (1) oxidizing two adjacent alcohol groups on the uronic acid of the glycosaminoglycan and ring-opening to form a dialdehyde structure, and (2) further oxidizing the dialdehyde structure to obtain a dicarboxylic acid structure, and
wherein the glycosaminoglycan is heparin or heparan sulfate.

3. The method of claim 1, wherein the compound of formula (I) is selected from the group consisting of the following structural formulas:

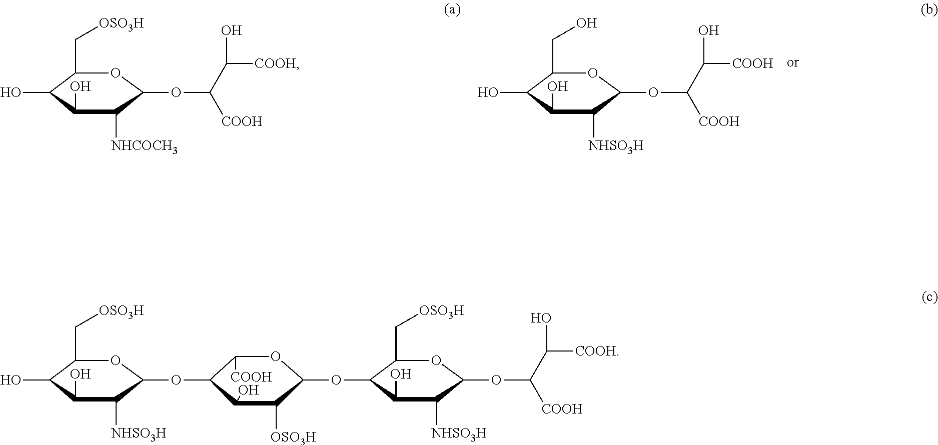

4. The method of claim 1, wherein the carboxylated glycosaminoglycan derivative has a weight average molecular weight of 3000-20000 Da, preferably 7000-14000 Da, and further preferably 8000-13500 Da;
the carboxylated glycosaminoglycan derivative has a ring-opening degree of 10-100%, preferably 25-80%, and further preferably 25-60%.

5. The method of claim 1, wherein in step (1), a method of hydrolyzing the sample containing a carboxylated glycosaminoglycan derivative is heating;
preferably, the heating is performed at 70-100° C., preferably 85-95° C.;
preferably, the heating is performed for 12-168 h, preferably 12-120 h.

6. The method of claim 1, wherein the liquid chromatography is reversed-phase chromatography, size-exclusion chromatography or hydrophilic chromatography;
preferably, mobile phases of the liquid chromatography are mobile phase A and mobile phase B; the mobile phase A is an aqueous solution of hexafluoroisopropanol and pentylamine; the mobile phase B is an acetonitrile-water solution of hexafluoroisopropanol and pentylamine;
preferably, the mobile phase A is an aqueous solution containing 45-55 mM hexafluoroisopropanol and 13-17 mM pentylamine; the mobile phase B is an acetonitrile-water solution containing 45-55 mM hexafluoroisopropanol and 13-17 mM pentylamine; the mobile phase B has a volume ratio of acetonitrile to water of 70:30-80:20;
preferably, the mobile phases of the liquid chromatography are mobile phase A and mobile phase B, which as below in the table.

| | |
|---|---|
| Mobile | 50 mM hexafluoroisopropanol, 15 mM pentylamine, H₂O |
| phase A | |
| Mobile | 50 mM hexafluoroisopropanol, 15 mM pentylamine, acetonitrile/ |
| phase B | H₂O (75/25, v/v) |

7. The method of claim 1, wherein, when the sample containing a carboxylated glycosaminoglycan derivative is a biological sample, the hydrolysate obtained in step (1) requires pretreatment before detection; the pretreatment comprises: mixing the hydrolysate with a trifluoroacetic acid solution and an acetonitrile-methanol solution, then performing standing and centrifugation, collecting a supernatant and drying, and then re-dissolving with water;
preferably, the biological sample comprises blood and urine;
preferably, the trifluoroacetic acid solution is added in an amount of 0.5-1.5% of a volume of the hydrolysate;
preferably, the trifluoroacetic acid solution has a concentration of 4-6%;
preferably, the acetonitrile-methanol solution is added in an amount of 1-5 times of a volume of the hydrolysate, preferably 1-3 times, and more preferably 1.5-2.5 times;
preferably, a volume ratio of acetonitrile to methanol in the acetonitrile-methanol solution is 1:0.5-1:1.5;
preferably, the standing is performed at −25 to −15° C. for 15-25 min

8. A compound, which has a structure of formula (I): 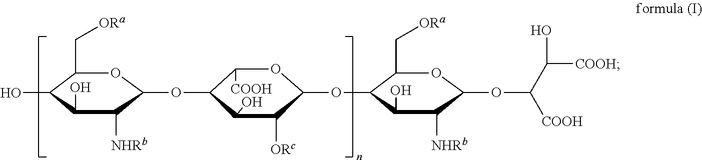
wherein, each R^{a} is independently —SO₃H or —H, each R^{b} is independently H, —SO₃H or —C(O)CH₃, each R^{c} is independently —SO₃H or —H, and n is 0, 1, 2, 3, 4 or 5.

9. The compound of claim 8, wherein the compound has one of the following structures:

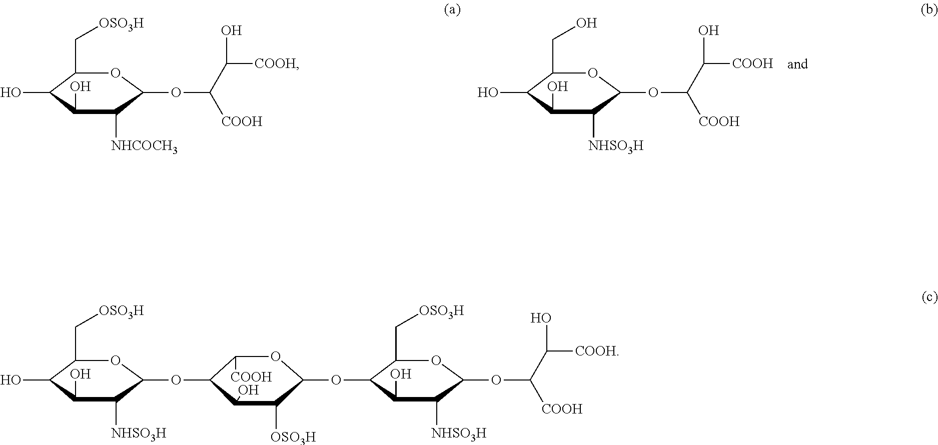

10.-11. (canceled)

12. A method of a pharmacokinetic study of a carboxylated glycosaminoglycan derivative comprising the method for detecting a carboxylated glycosaminoglycan derivative of claim 1.

13. A method of a quality test of a carboxylated glycosaminoglycan derivative pharmaceutical preparation comprising the method for detecting a carboxylated glycosaminoglycan derivative of claim 1.
